SEQUENCE LISTING (2 sequences)

SEQ ID NO: 1 - protein, 432 aa, Artificial Sequence
  Peptide
MLGNSAPGPATREAGSALLALQQTALQEDQENINPEKAAPVQQPRTRAALAVLKSGNPRGLAQQQRPKTRRVAPLKDLPVNDEHVTVPPWKANSKQPAFTIHVDEAEKEAQKKPAESQKIEREDALAFNSAISLPGPRKPLVPLDYPMDGSFESPHTMDMSIVLEDEKPVSVNEVPDYHEDIHTYLREMEVKCKPKVGYMKKQPDITNSMRAILVDWLVEVGEEYKLQNETLHLAVNYIDRFLSSMSVLRGKLQLVGTAAMLLASKFEEIYPPEVAEFVYITDDTYTKKQVLRMEHLVLKVLTFDLAAPTVNQFLTQYFLHQQPANCKVESLAMFLGELSLIDADPYLKYLPSVIAGAAFHLALYTVTGQSWPESLIRKTGYTLESLKPCLMDLHQTYLKAPQHAQQSIREKYKNSKYHGVSLLNPPETLNL

SEQ ID NO: 2 - DNA, 407 bp, Artificial Sequence
  Promoter
gcagtctgggctttcacaagacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacct

(19) United States
(12) Patent Application Publication
(10) Pub. No.: US 2021/0316015 A1
(43) Pub. Date: Oct. 14, 2021

(54) COMPOSITIONS AND METHODS FOR THE TREATMENT OF HEART DISEASE

(71) Applicant: Icahn School of Medicine at Mount Sinai, New York, NY (US)

(72) Inventor: Hina W. Chaudhry, New York, NY (US)

(21) Appl. No.: 17/273,805

(22) PCT Filed: Sep. 5, 2019
(86) PCT No.: PCT/US19/49694

§ 371 Date: Mar. 5, 2021

Related U.S. Application Data
(60) Provisional application No. 62/727,587, filed on Sep. 6, 2018.

(51) Int. Cl. A61K 48/00; A61K 38/18; C12N 15/86; C12N 7/00; A61K 35/34; A61P 9/04; A61K 9/00

(52) U.S. Cl. CPC A61K 48/0058; A61K 38/18; C12N 15/86; C12N 2750/00043; A61K 35/34; A61P 9/04; A61K 9/0029; C12N 7/00

(57) ABSTRACT

The disclosure relates to compositions and methods for promoting cardiomyocyte cytokinesis and cardiomyocyte proliferation and for use in cardiac regenerative therapy. Embodiments of the disclosure are particularly useful for promoting cytokinesis in adult cardiomyocytes. In embodiments, the disclosure relates to the expression of human cyclin A2 (CCNA2) under the control of a cardiac Troponin T (cTNT) promoter to promote cytokinesis of adult human cardiomyocytes.

### STATEMENT REGARDING FEDERALLY SPONSORED RESEARCH

This invention was made with government support under grant numbers HL067048, HL088255, and HL088867 awarded by the National Institutes of Health. The government has certain rights in the invention.

### BACKGROUND

### Field of the Invention

The present description relates generally to the field of molecular biology and medicine. More particularly, the methods and compositions herein are useful for promoting cell cycle regulation of heart cells for the treatment of cardiovascular disease.

### Description of Related Art

Heart disease remains a leading global cause of death in the industrialized world. The vast morbidity and mortality of heart disease is, in large part, attributed to the inability of adult human cardiomyocytes to divide in a clinically sufficient manner. Scar formation via fibrosis is therefore the primary response to cardiac injury. A multitude of molecular and cellular approaches have been investigated over the past 15 years aimed at regenerating the myocardium in various states of heart disease. Studies centered on the use of stem cells have remained controversial given the marginal results in regards to improvement in cardiac contractile function noted in clinical trials of cardiovascular cell therapy. Further, there has not been conclusive evidence in these trials that cell types utilized actually differentiate to functional cardiomyocytes.

One of the main challenges in using cardiac regeneration as a treatment of injury such as myocardial infarction arises from the observation that cardiomyocytes naturally exhibit very low levels of turnover in the healthy human heart. Such cardiomyocyte turnover is even more limited in aging individuals, the main demographic group with heart disease in industrialized countries (Mollova et al., Proc Natl Acad Sci USA. 2013 Jan. 22; 110(4):1446-51 and Bergmann et al., Science. 2009 Apr. 3; 324(5923):98-102).

Cyclin A2 (CCNA2) serves as a regulator of the cardiomyocyte cell cycle. Unlike other cyclins, cyclin A2 complexes with its cyclin-dependent kinase partners to mediate both of the two transitions of the cell cycle: G1-S and G2-M. CCNA2 is silenced shortly after birth in mammalian cardiomyocytes. It has previously been shown that expression of CCNA2 can mediate cardiac repair by inducing cardiomyocyte mitoses after myocardial infarction (MI) in rodents and pigs (Shapiro et al., Sci Transl Med. 2014 Feb. 19; 6(224):224ra27, Cheng et al., Circ Res. 2007 Jun. 22; 100(12):1741-8, and Woo et al., Circulation. 2006 Jul. 4; 114(1 Suppl):I206-13). However, such feat has—to the inventors' knowledge—never been achieved with adult human cardiomyocytes. Demonstrating efficacy in human vs non-human animals is critical, as mechanisms of cardiac repair may be widely divergent among species. As such, data in large animals from pigs, sheep, dogs, and even primates cannot perfectly predict efficacy of a given therapy in humans. As such, there remains a considerable need for compositions and methods that can promote cytokinesis and proliferation in cardiomyocyte of adult humans.

### SUMMARY OF THE INVENTION

The present disclosure relates the compositions and methods for promoting cardiomyocyte cytokinesis and cardiomyocyte proliferation and for use in cardiac regenerative therapy. Embodiments of the disclosure are particularly useful for promoting cytokinesis in adult cardiomyocytes.

In one embodiment, the disclosure relates to the expression of human CCNA2 under the control of a cardiac Troponin T (cTNT) promoter to promote cytokinesis of adult human cardiomyocytes.

In some embodiments, the disclosure relates to methods of treating an adult human subject with heart disease and/or a family history of heart disease by administering to that adult human subject a vector comprising a human CCNA2 gene under the control of a cardiac Troponin T (cTNT) promoter.

In certain embodiments, the patient to which the vector is administered has (i) heart failure or heart tissue damage or degeneration and/or (ii) a family history of heart failure or heart tissue damage or degeneration.

In some embodiments, the subject experiences cardiomyocyte hyperplasia, improved cardiac ejection fraction, and/or increased cardiac output after administration of the vector administered to the adult human subject.

In some embodiments, the vector administered to the adult human subject is a viral vector, and preferentially a replication-deficient adenovirus vector. In preferred embodiments, the vector administered to the adult human subject is an E1/E3 deleted adenovirus 5 vector.

In embodiments of the disclosure, the vector is administered to the adult human subject parenterally. In some embodiments, the vector is administered to the adult human subject by a catheter inserted into the adult human subject's heart tissue.

In certain embodiments, the adult human subject is over 20 years of age.

The disclosure further relates to methods of treating an adult human subject with heart disease and/or a family history of heart disease by

a) providing a population of heart tissue cells, side-population progenitor cells, or stem cells;
(b) transfecting the cells with a vector comprising a human CCNA2 gene under the control of a cardiac Troponin T (cTNT) promoter; and
(c) introducing the cells into the adult human subject.

In some of these embodiments, the human cyclin A2 protein is expressed in the cells (i) in vitro prior to introducing the cells into the adult human subject, (ii) in vivo after introducing the cells into the adult human subject, or (iii) both.

In certain embodiments, the adult human subject that is treated has (i) heart failure or heart tissue damage or degeneration and/or (ii) a family history of heart failure or heart tissue damage or degeneration.

In some embodiments, the adult human subject that is treated experiences cardiomyocyte hyperplasia, improved cardiac ejection fraction, and/or increased cardiac output after introducing the transfected cells into the adult human subject.

In some embodiments, the vector used for transfecting the cells is a viral vector, and preferentially a replication-deficient adenovirus vector. In preferred embodiments, the vector used for transfecting the cells is an E1/E3 deleted adenovirus 5 vector.

In certain embodiments, the adult human subject is over 20 years of age.

Also contemplated by the disclosure are therapeutic compositions comprising a human CCNA2 gene under the control of a cardiac Troponin T (cTNT) promoter.

In some embodiments, the therapeutic composition comprises a nucleic acid encoding a human cyclin A2 protein that comprises an amino acid sequence that is at least 90% similar to SEQ ID NO: 1. In a preferred embodiment, the therapeutic composition comprises a nucleic acid encoding a human cyclin A2 protein that has an amino acid sequence according to SEQ ID NO: 1.

In one aspect of the disclosure, the human CCNA2 gene is under the control of a promoter that comprises a nucleotide sequence that is at least 90% identical to SEQ ID NO: 2. In preferred aspects of the disclosure, the human CCNA2 gene is under the control of a promoter according to SEQ ID NO: 2.

Also contemplated by the disclosure are vectors comprising a nucleic acid encoding a human cyclin A2 protein that comprises an amino acid sequence that is at least 90% similar to SEQ ID NO: 1. In a preferred embodiment, the vector comprises a nucleic acid encoding a human cyclin A2 protein that has an amino acid sequence according to SEQ ID NO: 1.

In yet another embodiment of the disclosure, the vector comprises a human CCNA2 gene, which is under the control of a promoter that comprises a nucleotide sequence that is at least 85% identical to SEQ ID NO: 2. In preferred aspects of the disclosure, vector comprises a human CCNA2 gene, which is under the control of a promoter having a sequence according to SEQ ID NO: 2.

The disclosure also relates to methods of promoting cardiomyocyte cytokinesis and proliferation by

a) providing population of heart tissue cells, side-population progenitor cells, or stem cells; and
b) transfecting the cells with a vector comprising a nucleic acid encoding human cyclin A2 protein, wherein the nucleic acid is expressed under the control of a cardiac Troponin T promoter.

In certain embodiments, the transfection of the cells occurs in vitro, ex vivo, and/or in vivo.

In some embodiments, the vector used for transfecting the cells is a replication-deficient adenovirus vector, preferentially an E1/E3 deleted adenovirus 5 vector.

These and other features, aspects, and advantages of the present invention will become better understood with regard to the following description, appended claims and accompanying drawings.

### BRIEF DESCRIPTION OF THE FIGURES

FIG. 1 is a representation of the adenovirus vector construct. Clinical-use grade replication-deficient human adenovirus type 5 (Ad5) was used for this study. The vector was designed with cardiac troponin T (cTnT) promoter to express human cyclin A2 (hCCNA2) specifically in cardiomyocytes.

FIG. 2 illustrates cyclin A2 expression in adult human cardiomyocytes in vitro. The cTnT-hCCNA2 adenovirus was used to transfect cultured adult human cardiomyocytes to induce expression of human CCNA2. Significantly higher CCNA2 expression was observed in cells transfected with cTnThCCNA2 compared to control virus.

FIGS. 3A, 3B, 3C, and 3D illustrate that CCNA2 expression induces cytokinesis in adult human cardiomyocytes in vitro. FIG. 3A Still images from representative time lapse epifluorescence microscopy of cultured cardiomyocytes isolated from adult human (55-year-old male). At time 0 hr a cell (yellow) expressing both troponin Tc (green) and α-actinin (red) is shown to be followed for 70 hrs via time lapse microscopy. Cells were transfected with a) CCNA2-adenovirus then co-transfected with cTnt-eGFP (to label cardiomyocytes) and CMV-α-actinin-m-cherry adenoviruses or b) cTnt-eGFP and CMV-α-actinin-m-cherry adenoviruses (control group) before start of the time lapse imaging. After 0.0 hrs of imaging the green channel was closed and cells were only followed through red channel to avoid the UV photo-toxicity. The observed human cardiomyocytes show the 1st cell division at 50 hrs of imaging and one of the daughter cells again undergoes division at 70 hrs of imaging. In FIG. 3B one daughter cell at 70 hrs is partially magnified with grayscale version also to demonstrate the presence of intact sarcomere structure. FIG. 3C The cytokinetic events were enumerated in both control and test samples and cytokinetic index was plotted. A significantly higher rate of cytokinesis was observed in test samples compared to controls. FIG. 3D One day after live imaging ended, this well was fixed with subsequent labeling of nuclei with DAPI. The green fluorescence of the original cTNT-eGFP transfection is visible, further confirming these cells are cardiomyocytes. More green-fluorescing cells are visible than red-fluorescing cells in FIG. 3A as this is after cells were fixed and thus exposure time is greater than exposure time utilized in live imaging.

FIGS. 4A and 4B illustrate that CCNA2 expression induces cytokinesis in adult human cardiomyocytes in vitro. Experimental conditions were as described for FIGS. 3A, 3B, 3C, and 3D, except that the cardiomyocytes were isolated from a 41-year-old female (FIG. 4A) or a 21-year-old male (FIG. 4B).

### DETAILED DESCRIPTION

Unless defined otherwise, all technical and scientific terms used herein have the same meanings as commonly understood by one of ordinary skill in the art to which this invention belongs. All publications referred to herein are incorporated by reference in their entirety and are not admitted to be prior art with respect to the present invention by their mention. If a definition set forth in this section is contrary to or otherwise inconsistent with a definition set forth in the publications that are herein incorporated by reference, the definition set forth in this section prevails over the definition that is incorporated herein by reference.

### Definitions

Unless indicated otherwise, the terms below have the following meaning:

The singular forms “a,” “an,” and “the” include plural reference unless the context clearly dictates otherwise. Thus, for example, reference to “an” agent is a reference to one or more agents and equivalents thereof known to those skilled in the art, and so forth.

As used herein, the term “adult” means a human of 20 years or older, or a human whose cardiomyocytes have a rate of turnover comparable to the rate of turnover of cardiomyocytes derived from an individual of 20 years or older. Cellular “turnover”, as used herein, refers to the balance between cell proliferation and death that contributes to cell and tissue homeostasis. As a non-limiting example, cells of the heart and brain are characterized by low turnover/long lifespan, while other organs and tissues, such as the outer layers of the skin and blood cells, are maintained by high cell turnover rates/short lifespan.

As used herein, the term “amino acid sequence” refers to an oligopeptide, peptide, polypeptide, peptidomimetic or protein sequence, or to a fragment, portion, or subunit of any of these, and to naturally occurring or synthetic molecules contemplated by the disclosure, or a biologically active fragment thereof.

As used herein “codon optimization” relates to the process of altering a naturally occurring polynucleotide sequence to enhance expression in the target organism, for example, humans.

As used herein, the term “cytokinesis” or “cell division” refer to the phase of mitosis in which a cell undergoes cell division. In other words, it is the stage of mitosis in which a cell's partitioned nuclear material and its cytoplasmic material are divided to produce two daughter cells. The period of cytokinesis is identifiable as the period, or window, of time between when a constriction of the cell membrane (a “cleavage furrow”) is first observed and the resolution of that constriction event, i.e., the generation of two daughter cells.

As used herein the term cytokinetic index is measured by counting the numbers of cells visualized undergoing complete cytokinesis via time-lapse imaging and dividing this number by the total number of cardiomyocytes in that particular well/petri dish.

As used herein, the term “generation” includes the generation of new heart tissue and the regeneration of heart tissue where heart tissue previously existed.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree of similarity or identity between nucleic acid or amino acid sequences is a function of the number of identical or matching nucleotides or amino acids at shared positions. Various alignment algorithms and/or programs may be used to calculate the similarity and/or identity between two sequences, including FASTA or BLAST, and can be used with, e.g., default setting. For example, polypeptides having at least 70%, 85%, 90%, 95%, 98% or 99% identity to specific polypeptides described herein and preferably exhibiting substantially the same functions, as well as polynucleotides encoding such polypeptides, are contemplated.

As used herein “improving or enhancing cardiac function” refers to improving, enhancing, augmenting, facilitating or increasing the performance, operation or function of the heart and/or circulatory system of a subject. An improvement in cardiac function may be readily assessed and determined by the skilled artisan, based on known procedures, including but not necessarily limited to, measuring volumetric ejection fraction using MRI.

As used herein, the terms “patient”, “subject” and “individual” are used interchangeably and refer to a human presenting to a medical provider for diagnosis or treatment of a disease. A subject can be afflicted with or be susceptible to a disease or disorder but may or may not display symptoms of the disease or disorder.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The terms “proliferation” and “growth”, as used herein, refer to an increase in mass, volume, and/or thickness of heart tissue, as well as an increase in diameter, mass, or number of heart tissue cells.

As used herein, the term “promoting generation of heart tissue” includes activating, enhancing, facilitating, increasing, inducing, initiating, or stimulating the growth and/or proliferation of heart tissue, as well as activating, enhancing, facilitating, increasing, inducing, initiating, or stimulating the differentiation, growth, and/or proliferation of heart tissue cells. Thus, the term includes initiation of heart tissue generation, as well as facilitation or enhancement of heart tissue generation already in progress. “Differentiation” is the cellular process by which cells become structurally and functionally specialized during development.

As used herein, a “substantially identical” amino acid sequence also can include a sequence that differs from a reference sequence (e.g., an exemplary sequence of the disclosure, e.g., a protein comprising an amino acid selected of SEQ ID NOs. 1) by one or more conservative or non-conservative amino acid substitutions, deletions, or insertions, provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, resulting in modification of the structure of the polypeptide without significantly altering its biological activity. For example, amino- or carboxyl-terminal amino acids that are not required for activity of cyclin A2 can be removed. Similarly, a “substantially identical” nucleotide sequence also can include a sequence that differs from a reference sequence (e.g., an exemplary sequence of the disclosure, e.g., a promoter sequence comprising the nucleotide sequence of SEQ ID NOs. 2) by one or more nucleotide substitutions, deletions, or insertions, provided that the polynucleotide essentially retains its functional properties.

As used herein, the term “therapeutic” means an agent utilized to treat, combat, ameliorate, prevent, or improve an unwanted condition or disease of a patient.

The terms “treat,” “treated,” “treating” or “treatment” as used herein refer to both therapeutic treatment and prophylactic or preventative measures, wherein the object is to prevent or slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “vector” refers to a nucleic acid molecule capable of transporting another nucleic acid to which it has been linked. A “vector” in the present disclosure includes, but is not limited to, a viral vector, a plasmid, a RNA vector or a linear or circular DNA or RNA molecule which may consists of a chromosomal, non-chromosomal, semi-synthetic or synthetic nucleic acids. In some embodiments, the vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, and spumavirus.

It is to be understood that this invention is not limited to the particular molecules, compositions, methodologies, or protocols described, as these may vary. Any methods and materials similar or equivalent to those described herein can be used in the practice or testing of embodiments of the present disclosure. It is also to be understood that the terminology used in the description is for the purpose of describing the particular versions or embodiments only, and is not intended to limit the scope of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Methods and compositions contemplated by the disclosure relate to the expression of human CCNA2 for promoting human cardiomyocyte cytokinesis, a process that is naturally very limited in adult humans. As such, embodiments contemplated by the disclosure now provide the unique opportunity to enhance cardiac function in adult individuals, who constitute the vast majority of people with heart disease, as part of cardiac regenerative therapy.

In some embodiments, the expression of human CCNA2 induces cytokinesis of cardiomyocytes or an increased rate of cardiomyocyte proliferation, or both.

In some embodiments, a patient receiving cardiac regenerative therapy has developed, or has a propensity to develop, heart failure, heart tissue damage and/or heart tissue degeneration. Causes of heart tissue degeneration include, without limitation, chronic heart damage, chronic heart failure, damage resulting from injury or trauma, damage resulting from a cardiotoxin, damage from radiation or oxidative free radicals, damage resulting from decreased blood flow, and myocardial infarction (such as a heart attack). Preferably, the degenerated heart tissue of the present disclosure results from a myocardial infarction or heart failure. In certain embodiments, the patient has a family history of heart failure, heart tissue damage and/or heart tissue degeneration.

Embodiments contemplated by the disclosure may further be utilized for promoting generation, regeneration and/or repair of cardiac tissue, inducing endogenous myocardial regeneration, and/or preventing or treating heart failure in a subject in need thereof. Methods and compositions relating to the disclosure are particularly suitable for repopulating degenerated (damaged or injured) heart tissue in a subject, through either in vitro generation of heart tissue and subsequent transplant thereof into a patient or in vivo/in situ generation/regeneration of heart tissue. In the case of regeneration, the heart tissue cells of the present disclosure may be obtained from, or found within, damaged or degenerated heart tissue (i.e., heart tissue that exhibits a pathological condition).

In some embodiments, the patient experiences improved, enhanced, augmented, facilitated or increased performance, operation or function of the heart and/or circulatory system after receiving cardiac regenerative therapy. In preferred embodiments, the methods and compositions of the disclosure are used to achieve cardiomyocyte hyperplasia, improved cardiac ejection fraction and/or increased cardiac output.

As discussed above, the compositions and methods of the disclosure are useful for the generation of new heart tissue and regeneration of existing heart tissue. Generation and regeneration may be measured or detected by known procedures, including Western blotting for heart-specific proteins, electron microscopy in conjunction with morphometry, simple assays to measure rate of cell proliferation (including trypan blue staining, the CellTiter-Blue cell viability assay from Promega (Madison, Wis.), the MTT cell proliferation assay from ATCC, differential staining with fluorescein diacetate and ethidium bromide/propidium iodide, estimation of ATP levels, flow-cytometry assays, etc.), and any of the methods, molecular procedures, and assays disclosed herein.

The embodiments contemplated by the disclosure are particularly useful for the treatment of adult patients. In a preferred embodiment, the patient receiving treatment is at last 20 years of age. In some embodiments, the patient receiving treatment characteristic of a human of at least 20, 30, 40, 50, 60, 70, 80, 90, or 100 years of age.

### CCNA2 Genes and Proteins

In some embodiments, the disclosure relates to promoting cytokinesis or proliferation of cardiomyocytes by expressing human CCNA2 or a substantially identical protein. In a preferred embodiment, the amino acid sequence of human CCNA2 expressed to promote cytokinesis or proliferation in cardiomyoctyes comprises SEQ ID No: 1 (NCBI Reference Sequence: NM_001237.4; 374-1672 bp). In other embodiments, the amino acid sequence of the human CCNA2 protein expressed to promote cytokinesis or proliferation in cardiomyoctyes comprises a sequence with at least 85%-99% identity as compared to SEQ ID No: 1. In some embodiments, the expressed human CCNA2 protein comprises an amino acid sequences with at least 90%-99% similarity to a protein of SEQ ID No: 1. In some embodiments, the gene encoding the human CCNA2 protein is codon-optimized.

### Promoter for CCNA2 Expression

In some embodiments, human CCNA2 or a substantially identical protein is expressed under a cardiomyocyte-specific promoter such as the cardiac Troponin T (cTNT) promoter, or under the control of a substantially identical promoter. In a preferred embodiment, the promoter controlling human CCNA2 expression is a human cTNT promoter and has a nucleotide sequence that comprises SEQ ID NO: 2. In other embodiments, the human CCNA2 gene is under the control of a promoter comprising a nucleotide sequence with at least 85%-99% identity as compared to SEQ ID No: 2. The cardiac Troponin T (cTNT) promoter may be mutated to optimize the expression level of human CCNA2 and to achieve a desirable expression level.

### Cells Expressing Human CCNA2

In certain aspects of the disclosure, the human CCNA2 gene is introduced into cells that were isolated from a human subject. In one aspect of the disclosure, the isolated cells are reintroduced into the same, or into another human subject. In some aspects, human CCNA2 gene is introduced into cells in vivo (including in situ), ex vivo, and/or in vitro. Expression of the human CCNA2 gene may occur in vitro prior to introducing the genetically modified cells into an adult human subject, in vivo after introducing the cells into an adult human subject or both.

In preferred embodiments, the cells transfected with the human CCNA2 gene are derived from an adult human subject.

In certain embodiments, the transfected cells are heart tissue cells. As used herein, the term “heart tissue” includes, without limitation, the myocardium of the heart (including cardiac muscle fibers, connective tissue (endomysium), nerve fibers, capillaries, and lymphatics); the endocardium of the heart (including endothelium, connective tissue, and fat cells); the epicardium of the heart (including fibroelastic connective tissue, blood vessels, lymphatics, nerve fibers, fat tissue, and a mesothelial membrane consisting of squamous epithelial cells); and any additional connective tissue (including the pericardium), blood vessels, lymphatics, fat cells, and nervous tissue found in the heart. Cardiac muscle fibers are composed of chains of contiguous heart-muscle cells, or “cardiomyocytes”, joined end to end at intercalated disks. The heart tissue cells contemplated by the present disclosure may include progenitor cells (e.g., heart-tissue side-population progenitor cells) and differentiated or post-mitotic cells. The term “post-mitotic”, as used herein, refers to a cell that is in G0 phase (a quiescent state), and is no longer dividing or cycling.

In some embodiments, the transfected cells are side-population progenitor cells, which are derived from non-heart tissue (e.g., spleen, bone marrow, skeletal muscle, brain, liver, kidney, lung, small intestine, etc.).

In some embodiments, the transfected cells are stem cells, including, but not limited to hematopoietic stem cells, heart-derived stem cells, induced pluripotent stem cells, and embryonic stem cells.

In a preferred embodiment, the cells transfected with the human CCNA2 gene are human cardiomyocytes.

### Methods for the Genetic Engineering of Cells

Methods for introducing genetic material into cells are well known in the art. In one aspect of the disclosure, a human CCNA2 gene is introduced into the cells by absorption, electroporation, immersion, injection (including microinjection), introduction, liposome delivery, stem cell fusion (including embryonic stem cell fusion), transduction, transfection, transfusion, vectors, and other protein-delivery and nucleic-acid-delivery vehicles and methods.

In a preferred embodiment, the cells are transfected using a vector comprising a nucleotide sequence encoding a human CCNA2 gene, preferentially a viral vector. The viral vector may be an adeno-associated virus (AAV) vector or a derivative thereof. In one embodiment, the viral vector comprises an AAV genome from a naturally derived serotype, isolate or clade of AAV. In a preferred embodiment, the vector for the expression of human CCNA2 is a viral vector. In another preferred embodiment, the vector is a replication-deficient adenovirus vector, such as an E1/E3 deleted adenovirus 5 vector.

### Routes of Administration

In accordance with the method of the present disclosure, nucleotide encoding human CCNA2 may be administered to a human or animal subject by known procedures, including, without limitation, oral administration, parenteral administration, transdermal administration, and by way of a catheter. For example, the agent may be administered parenterally, by intracranial, intraspinal, intrathecal, or subcutaneous injection. The agent of the present disclosure also may be administered to a subject in accordance with any of the above-described methods for effecting in vivo contact between heart tissue cells or side-population progenitor cells and cyclin-associated agents. Preferably, the agent is administered to the subject by way of targeted delivery to heart tissue cells via a catheter inserted into the subject's heart.

For parenteral administration (i.e., administration by injection through a route other than the alimentary canal) or administration through a catheter, a cyclin-associated agent may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual, or by way of a catheter.

For transdermal administration, an agent may be combined with skin penetration enhancers, such as propylene glycol, polyethylene glycol, isopropanol, ethanol, oleic acid, N-methylpyrrolidone, and the like, which increase the permeability of the skin to the agent, and permit the agent to penetrate through the skin and into the bloodstream. The agent/enhancer composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch.

### Therapeutic Compositions

Also contemplated are therapeutic compositions for promoting cardiomyocyte cytokinesis or proliferation. In some aspects of the disclosure, the human CCNA2 gene is provided with a pharmaceutically-acceptable carrier, which must be “acceptable” in the sense of being compatible with the other ingredients of the composition, and not deleterious to the recipient thereof. The pharmaceutically-acceptable carrier employed herein can be selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include, without limitation, carboxymethyl cellulose, crystalline cellulose, glycerin, gum arabic, lactose, magnesium stearate, methyl cellulose, powders, saline, sodium alginate, sucrose, starch, talc, and water, among others. Formulations of the therapeutic composition of the present disclosure may be prepared by methods well-known in the pharmaceutical arts.

The human CCNA2 gene is provided in an amount that is effective to promote cytokinesis and/or proliferation of adult human cardiomyocytes, to improve or enhance cardiac function, to promote generation, regeneration and/or repair of cardiac tissue, to induce endogenous myocardial regeneration, and/or to prevent or treating heart failure heart tissue degeneration in a subject to whom the therapeutic composition is administered. This amount may be readily determined by the skilled artisan.

### EXAMPLES

### Example 1: Design of a Therapeutic Use Grade, Human CCNA2 Adenovirus Vector

This Example demonstrates that a cTnT-CCNA2 adenovirus vector can be used to induce expression of CCNA2 in cultured adult human cardiomyocytes.

### A. Methods

### I. Culture of Adult Human Cardiomyocytes

Cardiomyocytes from adult human heart tissue (55-year-old male who died of a non-cardiac cause) were isolated after enzymatic digestion at Anabios, San Diego, Calif. and were processed within 24 h. Adult human cardiomyocytes were subjected to the following culture conditions: Cells were washed with serum free DMEM media twice and 10⁵ cells were seeded in 100-mm untreated polystyrene plates (Fisher Scientific). Non-adherent cells were collected every 24 h and centrifuged at 20 g for 2 min at room temperature. The cell pellet was washed with serum-free DMEM and seeded on new polystyrene plates in modified Cardiomyocyte Culture Media22 (mod CMC) formulated by adding 13% FBS, 2.5% horse serum, lx nonessential amino acid, 1 mM sodium pyruvate, penicillin (100 U/ml), streptomycin (100 mg/ml), and fungizone (0.5 mg/ml) to Dulbecco's modified Eagle's medium (DMEM)/F12 (50:50). Cells were washed every day with plain media and re-seeded in new culture plated for 3 days. On day 4, the cells were seeded in glass-bottom 24-well tissue culture plates for 20 days with media changed every 4th day. The wells with cardiomyocyte adhesion and spreading were selected and cells were trypsinized, counted and 10³ cells per well were seeded in new glass bottom tissue culture plates.

### II. Transfection of Human Cardiomyocytes

After 2 days of culture, cells were divided into two groups and were transfected with adenoviruses. One group (test) was transfected with cTnT-hCCNA2 along with cTnT-eGFP (enhanced green fluorescent protein) and Adeno-act-mCherry adenoviruses while another group (control) was transfected with only cTnT-eGFP and Adeno-act-mCherry adenoviruses. The multiplicity of infection (MOI) of adenoviruses were adjusted to 180 in each well of test (with cTnt-CCNA2; MOI 100, Adeno-act-mCherry; MOI 40 and cTnt-GFP; MOI 40) and control (cTnt-GFP; MOI 140 and Adeno-act-mCherry; MOI 40) group. After 48 h of incubation, transfection was confirmed by observing desired fluores-cence in live cell imaging with Zeiss AxioVision Observer Z1 inverted microscope (Carl Zeiss).

### B. Results

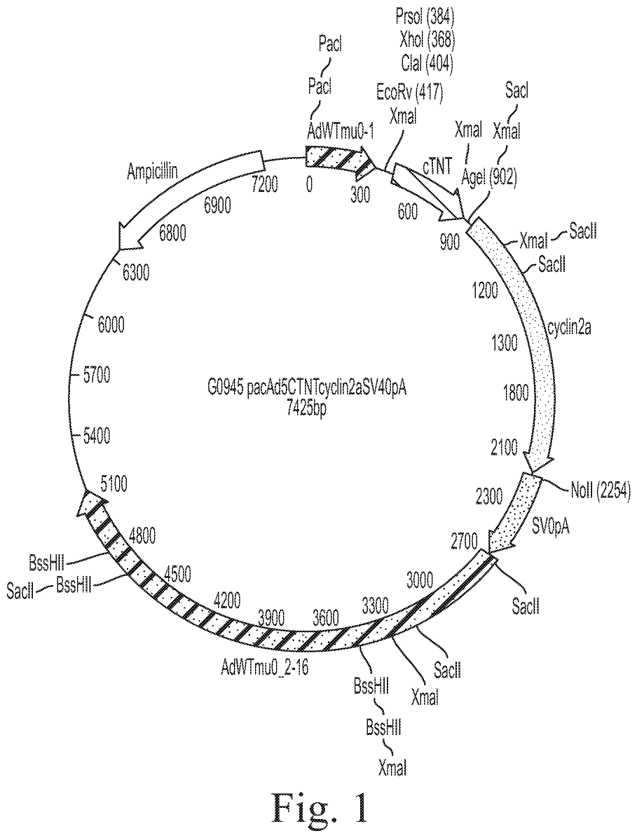

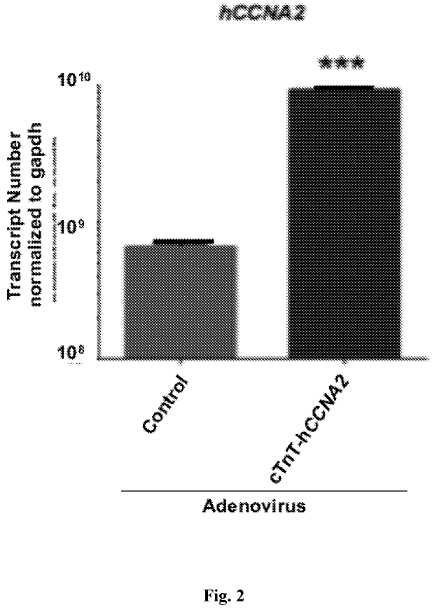

The therapeutic use grade human CCNA2 adenovirus vector was designed to express human CCNA2 (CCNA2) specifically in cardiomyocytes by cloning human cDNA downstream to the cardiac troponinT (cTnT) promoter (see FIG. 1). The cultured adult human cardiomyocytes were transfected with cTnT-CCNA2 (test) and cTnT-eGFP (control) adenoviruses with MOI of 100 each for assessing the induced expression of CCNA2. A significantly increased expression of CCNA2 in the cultured adult human cardiomyocytes transfected with test was observed compared to control adenovirus (see FIG. 2).

### Example 2: Use of a Therapeutic Use Grade, Human CCNA2 Adenovirus Vector for Promoting Cytokinesis in Adult Human Cardiomyocytes

This Example illustrates that adenoviral vector mediated expression of CCNA2 induces cytokinesis in cultured adult human cardiomyocytes.

### A. Methods

### I. Time-Lapse Microscopy

To capture cell division events in cardiomyocytes in vitro, live cell epifluorescence time-lapse microscopy were carried out using a Zeiss AxioVision Observer Z1 (Carl Zeiss, Thornwood, N.Y., USA) inverted epifluorescence microscope in a humidified chamber in the presence of 5% CO2 at 37° C. Multiple random points with cells expressing eGFP (green) and mCherry (red) were selected in the test and control groups. The positions were marked with the “position-list” tool in the AxioVision microscopy software (AxioVision Release 4.7, Carl Zeiss). After the first cycle of imaging, only the channel for Texas red was used (for detection of mCherry) to acquire images for 72 h. The fluorescein isothiocyanate (green) channel of the microscope was kept closed during the time-lapse imaging to avoid cell death from exposure to ultraviolet rays in this channel. Images were taken at intervals of 30 min. The objective lens of 10× was used for all time-lapse imaging. Time-lapse movies were generated after the end of each experiment and exported as .MOV files. The time lapse movies were analyzed and cells underwent successful cytokinesis were enumerated in each group. The % cytokinesis events were calculated for each position and graph was plotted.

### II. Cell Fixation and Nuclear Staining

After time-lapse microscopy, cells in the glass-bottom plate were fixed with 4% paraformaldehyde at room temperature for 20 min and were stored in 4° C. For nuclear staining, cells were washed with 1×PBS and permeabilized by incubating them in 0.5% Triton X-100 solution for 20 min at room temperature. Cells were washed three times with 1×PBS and incubated in DAPI solution (2.5 μg/ml) for 5 min. Cells were washed twice with 1×PBS and imaging was carried out by using a Zeiss AxioVision Observer Z1 inverted epifluorescence microscope.

### III. Real-Time Quantitative PCR

Quantitative PCR experiments were performed by using “SYBR Green quantitative PCR protocol” on the “StepOnePlus” real-time PCR system (Applied Biosystems, CA). The PCR protocol consisted of 40 cycles at 95° C. (15 s) and 60° C. (1 min). Gene expression was determined by using the formula 2\hat{ }(39−ΔΔCT) with consideration of CT value 39 for single transcript and with normalization to the endogenous control graph.

### B. Results

To assess the effect of induced CCNA2 expression on cell division in the adult human cardiomyocytes, cytokinesis in cultured, adult human cardiomyocytes was assessed in vitro using live cell epifluorescence time lapse microscopy. The adult human cardiomyocytes were plated at equal densities and transfected with cTnT-CCNA2 and cTnT-eGFP (test) or with cTnT-eGFP only (control). cTnT-eGFP adenovirus was transfected in both the groups to confirm the initial tracking of cardiomyocytes (green) during live cell epifluorescence microscopy. For delineation of sarcomeric structure in cardiomyocytes, cells from both groups were co-transfected with adenovirus containing α-actinin-mCherry, which was constructed to allow for proper folding of the virally delivered α-actinin into the live cardiomyocyte sarcomere (Adeno-act-mCherry). This strategy allows for a confirmation of cardiomyocyte identity by assessing the expression of eGFP before and after cytokinesis and tracking of sarcomere dynamics during live cell imaging. This strategy is more accurate than antibody-based identification, which can result in artifact and can only be used at one time point after cell fixation.

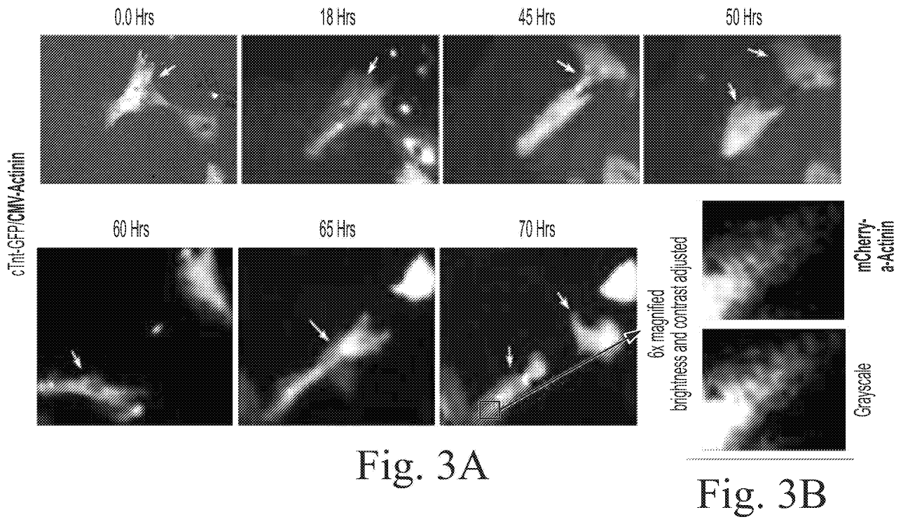

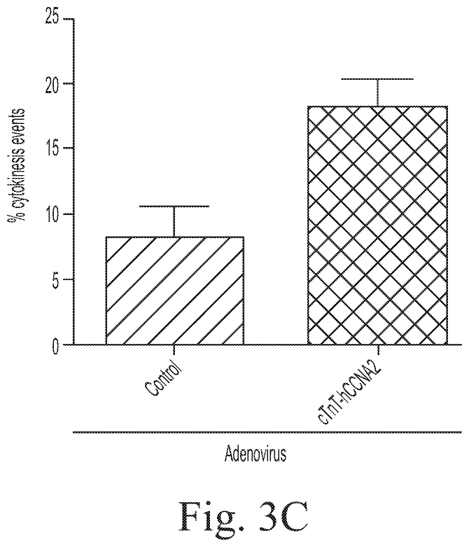

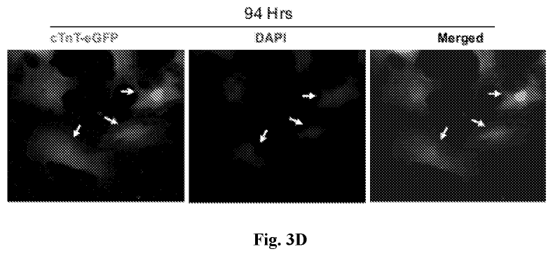

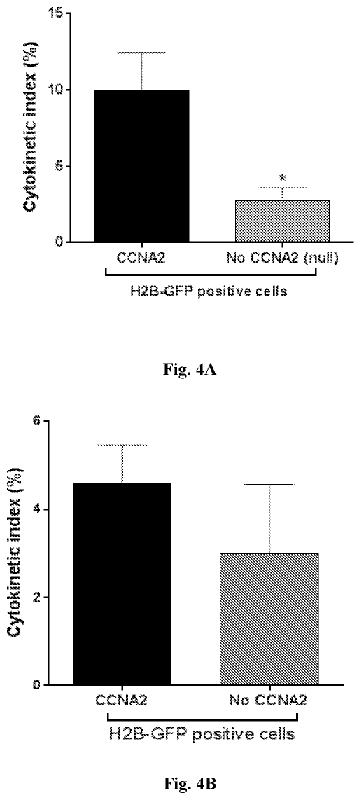

Co-expression of eGFP (green) and α-actinin (red) was observed in cultured adult cardiomyocytes (FIG. 3A; first panel). Time-lapse microscopic imaging of live cells was performed to capture cardiomyocyte cytokinesis (FIG. 3 A; remaining panels). The cytokinetic index of adult human cardiomyocytes was calculated by counting cytokinetic events observed in 42 regions of interest (ROIs) (Table 1). The cytokinetic index was significantly higher in the test samples with cTnT-CCNA2 adenovirus transfection compared to control samples (FIG. 3C). This was true for cardiomyocytes isolated from two different patients (55 year old male, FIG. 3C, and 41 year old female, FIG. 4A). Most remarkably, sarcomere structure was preserved in the daughter cells after cytokinesis (FIG. 3B; upon magnification of a daughter cell, the presence of sarcomeric structure is easily noted). The daughter cells were further identified with the expression of eGFP (as they were originally also transfected with cTNT-eGFP) and noted to be mononuclear after they had been fixed and stained with DAPI (FIG. 3D). Clusters of other cardiomyocytes with expression of eGFP that had not taken up the Adeno-actmCherry could be seen adjacent to the daughter cells.

Cardiomyocytes isolated from a 21 year old individual responded only mildly to expression of CCNA2 (FIG. 4B), possibly because cells that still retain a reasonable rate of turnover are less receptive to perturbation of the cell cycle as compared to cardiomyocytes in which cardiomyocyte cytokinesis is essentially completely silenced (FIG. 3C and FIG. 4A).

The cytokinetic index can also be used to estimate the proliferation rate of cardiomyocytes, another relevant marker of cellular turnover.

The low rate of cytokinetic activity observed in the control wells (See FIGS. 3C, 4A, and 4B) is likely caused by a reactivation of endogenous CCNA2 during the prolonged culture of cardiomyocytes. This phenomenon is likely not correlated with the low turnover seen in human hearts as measured by C14 dating (Bergmann et al., Science. 2009 Apr. 3; 324(5923):98-102) as cytokinesis was not noted at all in human hearts over the age 20 (Mollova et al., Proc Natl Acad Sci USA. 2013 Jan. 22; 110(4):1446-51).

TABLE 1

| Test (cTNT-CCNA2 + cTNT-eGFP) | | | | Control (cTNT-eGFP only) | | | |
|---|---|---|---|---|---|---|---|
| | Number | Cytokinetic | | | Number | Cytokinetic | |
| ROI | of cells | events | (%) | ROI | of cells | events | (%) |
| ROI 1 | 23 | 5 | 21.7 | ROI 29 | 13 | 1 | 7.7 |
| ROI 2 | 7 | 1 | 14.3 | ROI 30 | 33 | 1 | 3.0 |
| ROI 3 | 17 | 4 | 23.5 | ROI 31 | 21 | 1 | 4.8 |
| ROI 4 | 24 | 7 | 29.2 | ROI 32 | 9 | 3 | 33.3 |
| ROI 5 | 12 | 5 | 41.7 | ROI 33 | 27 | 3 | 11.1 |
| ROI 6 | 11 | 2 | 18.2 | ROI 34 | 33 | 1 | 3.0 |
| ROI 7 | 20 | 4 | 20.0 | ROI 35 | 29 | 0 | 0.0 |
| ROI 8 | 44 | 17 | 38.6 | ROI 36 | 20 | 1 | 5.0 |
| ROI 9 | 26 | 7 | 26.9 | ROI 37 | 16 | 0 | 0.0 |
| ROI 10 | 30 | 6 | 20.0 | ROI 38 | 30 | 5 | 16.7 |
| ROI 11 | 29 | 8 | 27.6 | ROI 39 | 28 | 1 | 3.6 |
| ROI 12 | 32 | 10 | 31.3 | ROI 40 | 16 | 0 | 0.0 |
| ROI 13 | 37 | 6 | 16.2 | ROI 41 | 17 | 2 | 11.8 |
| ROI 14 | 26 | 6 | 23.1 | ROI 42 | 16 | 2 | 12.5 |
| ROI 15 | 18 | 1 | 5.6 | | | Average | 8.0 |
| ROI 16 | 47 | 2 | 4.3 | | | | |
| ROI 17 | 40 | 1 | 2.5 | | | | |
| ROI 18 | 19 | 3 | 15.8 | | | | |
| ROI 19 | 18 | 2 | 11.1 | | | | |
| ROI 20 | 20 | 2 | 10.0 | | | | |
| ROI 21 | 22 | 2 | 9.1 | | | | |
| ROI 22 | 34 | 7 | 20.6 | | | | |
| ROI 23 | 27 | 3 | 11.1 | | | | |
| ROI 24 | 21 | 2 | 9.5 | | | | |
| ROI 25 | 9 | 1 | 11.1 | | | | |
| ROI 26 | 14 | 2 | 14.3 | | | | |
| ROI 27 | 15 | 2 | 13.3 | | | | |
| ROI 28 | 23 | 2 | 8.7 | | | | |
| | | Average | 17.8 | | | | |

| Sequences | |
|---|---|
| SEQ ID NO: 1 - human cyclin A2 | |
| 1 | MLGNSAPGPA TREAGSALLA LQQTALQEDQ ENINPEKAAP VQQPRTRAAL AVLKSGNPRG |
| 61 | LAQQQRPKTR RVAPLKDLPV NDEHVTVPPW KANSKQPAFT IHVDEAEKEA QKKPAESQKI |
| 121 | EREDALAFNS AISLPGPRKP LVPLDYPMDG SFESPHTMDM SIVLEDEKPV SVNEVPDYHE |
| 181 | DIHTYLREME VKCKPKVGYM KKQPDITNSM RAILVDWLVE VGEEYKLQNE TLHLAVNYID |
| 241 | RFLSSMSVLR GKLQLVGTAA MLLASKFEEI YPPEVAEFVY ITDDTYTKKQ VLRMEHLVLK |
| 301 | VLTFDLAAPT VNQFLTQYFL HQQPANCKVE SLAMFLGELS LIDADPYLKY LPSVIAGAAF |
| 361 | HLALYTVTGQ SWPESLIRKT GYTLESLKPC LMDLHQTYLK APQHAQQSIR EKYKNSKYHG |
| 421 | VSLLNPPETL NL |
| SEQ ID NO: 2 - cardiac Troponin T (cTNT) promoter | |
| 1 | GCAGTCTGGG CTTTCACAAG ACAGCATCTG GGGCTGCGGC AGAGGGTCGG GTCCGAAGCG |
| 61 | CTGCCTTATC AGCGTCCCCA GCCCTGGGAG GTGACAGCTG GCTGGCTTGT GTCAGCCCCT |
| 121 | CGGGCACTCA CGTATCTCCG TCCGACGGGT TTAAAATAGC AAAACTCTGA GGCCACACAA |
| 181 | TAGCTTGGGC TTATATGGGC TCCTGTGGGG GAAGGGGGAG CACGGAGGGG GCCGGGGCCG |
| 241 | CTGCTGCCAA AATAGCAGCT CACAAGTGTT GCATTCCTCT CTGGGCGCCG GGCACATTCC |
| 301 | TGCTGGCTCT GCCCGCCCCG GGGTGGGCGC CGGGGGGACC TTAAAGCCTC TGCCCCCCAA |
| 361 | GGAGCCCTTC CCAGACAGCC GCCGGCACCC ACCGCTCCGT GGGACCT |

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A method of treating an adult human subject, the method comprising:
a. administering a vector comprising a nucleic acid encoding human cyclin A2 protein to an adult human subject, wherein the nucleic acid is expressed under the control of a cardiac Troponin T promoter; and
wherein the adult human subject has (i) heart failure or heart tissue damage or degeneration and/or (ii) a family history of heart failure or heart tissue damage or degeneration.

2. The method according to claim 1, wherein the vector is a viral vector.

3. The method according to claim 2, wherein the viral vector is a replication-deficient adenovirus vector.

4. The method according to claim 3, wherein the replication-deficient adenovirus vector is an E1/E3 deleted adenovirus 5 vector.

5. (canceled)

6. The method according to claim 1, wherein the vector is administered to the adult human subject parenterally.

7. The method according to claim 1, wherein the vector is administered to the adult human subject by a catheter inserted into the adult human subject's heart tissue.

8. The method according to claim 1, wherein the adult human subject is over 20 years of age.

9. The method of treating an adult human subject, the method comprising:
(a) providing a population of heart tissue cells, side-population progenitor cells, or stem cells;
(b) transfecting the cells with a vector comprising a nucleic acid encoding human cyclin A2 protein, wherein the nucleic acid is expressed under the control of a cardiac Troponin T promoter; and
(c) introducing the cells into the adult human subject;
wherein the human cyclin A2 protein is expressed in the cells (i) in vitro prior to introducing the cells into the adult human subject (ii) in vivo after introducing the cells into the adult human subject, or (iii) both; and
wherein the adult human subject has (i) heart failure or heart tissue damage or degeneration and/or (ii) a family history of heart failure or heart tissue damage or degeneration.

10. The method according to claim 9, wherein the vector is a viral vector.

11. The method according to claim 10, wherein the viral vector is a replication-deficient adenovirus vector.

12. The method according to claim 11, wherein the replication-deficient adenovirus vector is an E1/E3 deleted adenovirus 5 vector.

13. (canceled)

14. The method according to claim 11, wherein the adult human subject is over 20 years of age.

15. A composition comprising vector comprising a nucleic acid encoding a human cyclin A2 protein, wherein the nucleic acid is expressed under the control of a cardiac Troponin T promoter.

16. The composition of claim 15, wherein the human cyclin A2 protein comprises an amino acid sequence that is at least 90% similar to SEQ ID NO: 1.

17. The composition of claim 15, wherein the human cyclin A2 protein has an amino acid sequence according to SEQ ID NO: 1.

18. The composition of claim 15, wherein the cardiac Troponin T promoter comprises a nucleotide sequence that is at least 90% identical to SEQ ID NO: 2.

19. The composition of claim 15, wherein the cardiac Troponin T promoter comprises a nucleotide according to SEQ ID NO: 2.

20. (canceled)

21. (canceled)

22. (canceled)

23. (canceled)

24. (canceled)

25. The composition according to claim 15, wherein the vector is a viral vector.

26. The composition according to claim 25, wherein the viral vector is a replication-deficient adenovirus vector.

27. The composition according to claim 26, wherein the replication-deficient adenovirus vector is an E1/E3 deleted adenovirus 5 vector.

28. A method of promoting cardiomyocyte cytokinesis and/or cardiomyocyte proliferation, the method comprising:
a. providing population of heart tissue cells, side-population progenitor cells, or stem cells; and
b. transfecting the cells with a vector comprising a nucleic acid encoding human cyclin A2 protein, wherein the nucleic acid is expressed under the control of a cardiac Troponin T promoter.

29. The method according to claim 28, wherein the transfection of the cells occurs in vitro.

30. The method according to claim 28, wherein the transfection of the cells occurs in vivo.

31. The method according to claim 28, wherein the transfection of the cells occurs ex vivo.

32. The method according to claim 28, wherein the vector is a viral vector.

33. The method according to claim 32, wherein the viral vector is a replication-deficient adenovirus vector.

34. The method according to claim 33, wherein the replication-deficient adenovirus vector is an E1/E3 deleted adenovirus 5 vector.
